(19) United States
(12) Patent Application Publication
(10) Pub. No.: US 2024/0010602 A1
(43) Pub. Date: Jan. 11, 2024

(54) PHENYL UNSATURATED KETONES AS HUMAN FACTOR XIIIA INHIBITORS FOR TREATMENT OF THROMBOEMBOLIC DISEASES

(71) Applicant: Xavier University of Louisiana, New Orleans, LA (US)

(72) Inventor: Rami A. AL-HORANI, New Orleans, LA (US)

(21) Appl. No.: 18/347,724

(22) Filed: Jul. 6, 2023

Related U.S. Application Data
(60) Provisional application No. 63/359,406, filed on Jul. 8, 2022.

(51) Int. Cl. C07C 59/90; C07C 69/734; C07C 235/20; A61P 7/02

(52) U.S. Cl. CPC C07C 59/90; C07C 69/734; C07C 235/20; A61P 7/02

(57) ABSTRACT

The present disclosure is directed to compounds having Formula (I) and their enantiomers, wherein the definitions of R, Y¹, Y², W, X, Z, and n are provided in the disclosure. The disclosure is also directed to pharmaceutical compositions of the disclosed compounds, as well as their use for treatment of thrombosis and related conditions and complications.

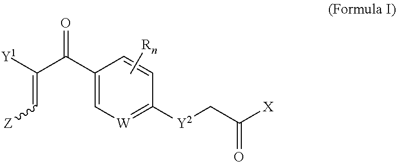

### CROSS REFERENCE TO RELATED PATENT APPLICATIONS

This Patent Application claims priority to U.S. Provisional Patent Application No. 63/359,406, filed on Jul. 8, 2022, the disclosure of which is incorporated herein in its entirety.

### STATEMENT REGARDING FEDERALLY SPONSORED RESEARCH OR DEVELOPMENT

This invention was made with government support under a grant from the National Institute of General Medical Sciences of National Institute of Health which has the number of 5SC3GM131986. The government has certain rights in the invention.

### BACKGROUND

### Field

The present disclosure relates to Factor XIIIa inhibitors and methods of use for the treatment of thromboembolic diseases.

### Description of Related Art

Factor XIII or fibrin stabilizing factor is a zymogen found in blood of humans and some other animals. It is activated by thrombin to factor XIIIa. Factor XIIIa is an enzyme of the blood coagulation system that crosslinks fibrin. Deficiency of factor XIII worsens clot stability and increases bleeding tendency. Human XIII is a heterotetramer. It consists of 2 enzymatic A peptides and 2 non-enzymatic B peptides. Factor XIIIa is a dimer of activated A peptides.

Human factor XIIIa (Factor XIIIa) catalyzes the last step in the coagulation process. It stabilizes the blood clot by cross-linking the α- and γ-chains of fibrin. It also protects the newly formed clot from plasmin-mediated fibrinolysis, primarily by cross-linking α₂-antiplasmin to fibrin. Furthermore, factor XIIIa is a major determinant of clot size and clot's red blood cells content.

Anticoagulants are prescribed to treat and prevent thromboembolic diseases. The inhibition of one or more coagulation proteins can be expected to reduce or prevent clot formation. Among all proteins in the coagulation cascade, the common pathway enzymes, thrombin and factor Xa, have been targeted. Mechanistically, anticoagulants utilize either an indirect or a direct mechanism for inhibiting these two enzymes. The direct inhibition mechanism targets the coagulation enzyme by binding to its active site or an allosteric site. The indirect inhibition mechanism involves recruiting a mediator protein, such as antithrombin, to reduce the proteolytic activity of coagulation enzymes. Structurally, anticoagulants could be: (1) Saccharide-based (unfractionated heparin, low molecular weight heparins, and fondaparinux); (2) Coumarin-based (warfarin), peptide-based (bivaluridin), or peptidomemtics (argatroban); (3) Peptidomemtics such as dabigatran and rivaroxaban that have been recently approved for clinical use as they directly inhibit thrombin and factor Xa, respectively, with high potency and substantial selectivity. However, current anticoagulants are associated with significant risk of bleeding which limits their use. There exists a need in the art for new anticoagulants.

### BRIEF SUMMARY

In an embodiment, a compound can be of Formula I:

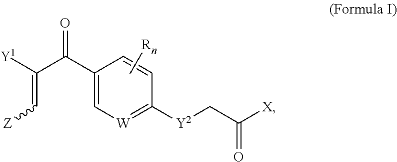

wherein, independently, R is —H, -halogen, C₁-C₆ alkyl, C₃-C₆ cycloalkyl, —OR³ (wherein R³═C₁-C₆ alkyl), -acyl, or -benzoyl;
n is an integer of 1 to 3;
Y¹ is C₁-C₁₂ alkyl, or H;
Y² is CH₂—, CH—(CH₃), C(CH₃)₂, NH, N(R⁴) (wherein R⁴ is C₁-C₆ alkyl), O, S, or C═O;
X is —OH, OR⁵ (wherein R⁵ is C₁-C₆ alkyl), NH₂, NHR⁶ (wherein R⁶ is C₁-C₆ alkyl), NR⁷R⁸ (wherein R⁷ and R⁸, independently, are C₁-C₆ alkyl), -halogen, or C₁-C₆ alkyl;
Z is H, —Cl, or C₁-C₆ alkyl (including E and Z-isomers); and
W is C, N, N(R⁹) (wherein R⁹ is C₁-C₆ alkyl), O, or S.

In an embodiment, a compound can be of Formula II:

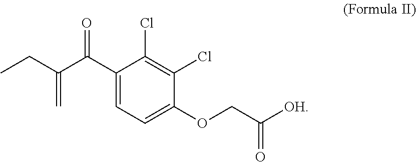 [SMILES: C=C(CC)C(=O)c1ccc(OCC(=O)O)c(Cl)c1Cl]

In an embodiment, a compound can be Formula III:

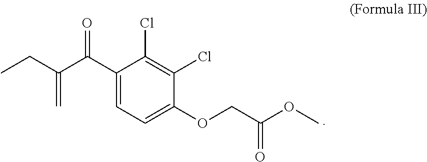 [SMILES: C=C(CC)C(=O)c1ccc(OCC(=O)OC)c(Cl)c1Cl]

In an embodiment, a compound can be of Formula IV:

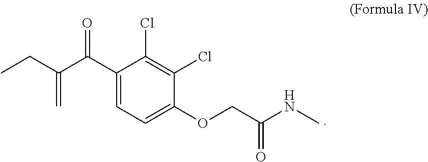 [SMILES: C=C(CC)C(=O)c1ccc(OCC(=O)NC)c(Cl)c1Cl]

In an embodiment, a compound can be of Formula V:

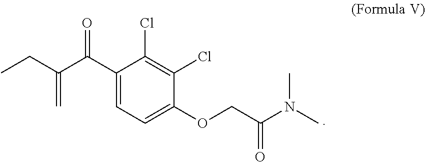 [SMILES: C=C(CC)C(=O)c1ccc(OCC(=O)N(C)C)c(Cl)c1Cl]

In an embodiment, a compound can be of Formula VI:

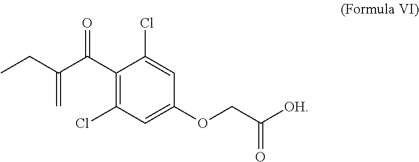 [SMILES: C=C(CC)C(=O)c1c(Cl)cc(OCC(=O)O)cc1Cl]

In an embodiment, a compound can be of Formula VII:

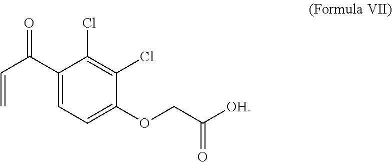 [SMILES: C=CC(=O)c1ccc(OCC(=O)O)c(Cl)c1Cl]

In an embodiment, a compound can be of Formula VIII:

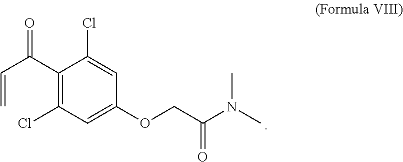 [SMILES: C=CC(=O)c1c(Cl)cc(OCC(=O)N(C)C)cc1Cl]

In an embodiment, a composition comprising a compound of Formula I, II, III, IV, V, VI, VII, VIII, or a combination thereof. The composition can be a pharmaceutical composition. The composition can further comprise a pharmaceutically acceptable excipient, carrier, diluent, adjuvant, vehicle, or a combination thereof.

In an embodiment, a method for treating thrombosis can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating thromboembolism can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need. The thromboembolism can be venous thromboembolism or arterial thromboembolism.

In an embodiment, a method for treating complications from chronic kidney disease can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating complications in acute coronary syndrome can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating Alzheimer's disease can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating cerebral amyloid angiopathy can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating acute kidney injury can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating acute lung injury can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, a method for treating coronary artery disease can comprise administering an effective amount of the compound of Formula I, II, III, IV, V, VI, VII, VIII, or a combination thereof, or a composition thereof, optionally further comprising a pharmaceutically acceptable excipient, carrier, diluent, adjuvant, vehicle, or a combination thereof, to a patient in need.

In an embodiment, the compound or composition can be administered intravenously, inhalation, subcutaneously, via infusion, orally, intrathecally, intraperitoneally, parenterally, or a combination thereof.

In an embodiment, the patient is a critically ill patient.

In an embodiment, the effective amount is between about 1 and 1,000 mg. The effective amount can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg.

In an embodiment, the Factor XIIIa inhibitors described herein can be used in a method for treating a condition selected from the group consisting of thrombosis, thromboembolism, complications from chronic kidney disease, complications in acute coronary syndrome, Alzheimer's disease, cerebral amyloid angiopathy, acute kidney injury, acute lung injury, coronary artery disease, or a combination thereof comprising administering an effective amount of the Factor XIIIa inhibitors described herein.

In an embodiment, the Factor XIIIa inhibitors described herein can be used in a method for treating a condition selected from the group consisting of thrombosis, thromboembolism, complications from chronic kidney disease, complications in acute coronary syndrome, Alzheimer's disease, cerebral amyloid angiopathy, acute kidney injury, acute lung injury, coronary artery disease, or a combination thereof comprising administering a composition comprising effective amount of the Factor XIIIa inhibitors described herein.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 depicts a general scheme of chemical synthesis. Example synthesis of inhibitor (I) with: disubstituted ring R is Cl—, Cl—; n is 2; Y¹ is —CH₂CH₃; X is OH; and Y² is O. a) Butanoyl-chloride, AlCl₃, S₂C; b) Ethyl bromoacetate, K₂CO₃, reflux, acetone; and c) C(O)—H₂, K₂CO₃, water/ethanol (1:1) reflux.

FIG. 2 depicts examples of factor XIIIa inhibition and the effect on human plasma clotting times of synthesized phenyl unsaturated ketones. The data is for the compound of Formula I.

FIG. 3 depicts examples of inhibition of fibrin(ogen) polymerization and inhibition fibrin(ogen)-α2-antiplasmin complex formation of synthesized phenyl unsaturated ketones. The data is for the compound of Formula I.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“Treatment” refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

“Effective amount,” as used herein, refers broadly to the amount of a compound, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

### Anticoagulants

Blood clotting prevents the excessive blood loss from tissue damage. Under normal physiological conditions, a balance is maintained between blood flow and blood clot, dysfunction of which may yield either hemorrhage or thrombosis. The enzymes'-mediated coagulation cascade controls the blood status. This cascade comprises mainly two pathways: the intrinsic pathway which is triggered by damage to blood vessel walls and the subsequent interactions with non-physiological surfaces such as collagen, lipoproteins, or bacteria, and the extrinsic pathway which is initiated by endothelium damage or hypoxia resulting from reduced blood flow. The two pathways converge at factor Xa. Factor Xa cleaves prothrombin to thrombin which further cleaves fibrinogen to form fibrin monomers. Factor XIIIa polymerizes fibrin monomers leading to the formation of the three-dimensional network of fibrin chains in the clot.

Thromboembolic diseases such as deep vein thrombosis, pulmonary embolism, stroke, and myocardial infarction are all triggered by formation of the pathological clot. Thromboembolic diseases are still amongst the most frequent causes of death not only in the US, but also all over the globe. Blood clotting disorders afflict a large number of people. For example, venous thromboembolism, which includes deep vein thrombosis and pulmonary embolism, affects annually about 600,000 individuals in the US. Thrombotic disorders are the second most frequent cause of death in cancer patients with an average mortality rate of nearly 18% of hospitalized cancer patients. Moreover, the prevalence of venous thromboembolism significantly varies among ethnic and racial groups. In the US, African Americans are more likely to be diagnosed with venous thromboembolism than any other group which further complicates the issue of health disparities.

Anticoagulants are prescribed to treat and prevent thromboembolic diseases. The inhibition of one or more coagulation proteins can be expected to reduce or prevent clot formation. Among all proteins in the coagulation cascade, the common pathway enzymes, thrombin and factor Xa, have been successfully targeted. Mechanistically, anticoagulants utilize either an indirect or a direct mechanism for inhibiting these two enzymes. The direct inhibition mechanism targets the coagulation enzyme by binding to its active site or an allosteric site. The indirect inhibition mechanism involves recruiting a mediator protein, such as antithrombin, to reduce the proteolytic activity of coagulation enzymes. Structurally, anticoagulants could be saccharide-based (unfractionated heparin, low molecular weight heparins, and fondaparinux), coumarin based (warfarin), peptide-based (bivaluridin), or peptidomemtics (argatroban).

Despite the clinical efficacy of the current anticoagulants, they are associated with serious drawbacks. Garcia et al. (2012) Chest 141(2 Suppl): e24S-43S; Ageno et al. (2012) Chest 141(2 Suppl): e44S-88S; and Weitz et al. (2012) Chest 141(2 Suppl): e120S-51S. Unfractionated heparin (UFH) suffers from significant patient response variation, which requires frequent laboratory monitoring. Heparin-induced thrombocytopenia is a lethal consequence of heparin therapy. Additional limitations of UFH include the development of osteoporosis in patients receiving heparin therapy for extended periods of time as well as the high risk of contamination with other glycosaminoglycans which may result in serious hypersensitivity reactions. UFH's drawbacks have been partially reduced by the introduction of low molecular weight heparins and fondaparinux. Warfarin suffers from a narrow therapeutic index and several drug-drug and drug-food interactions. Although the safety profiles of newer oral orthosteric inhibitors of thrombin and factor Xa are better than those of heparins and warfarin, yet the lack of standardized assays for measuring these drugs in biological fluids, their high cost, and potential contraindications in patients with severe renal dysfunction represent serious challenges for their therapeutic use. Many of direct anticoagulants are also P-glycoprotein substrates which carry significant drug-drug interaction issues. Nearly all direct anticoagulants require hepatic metabolism for elimination which affects their use in patients with hepatic dysfunction. Importantly, all anticoagulants are associated with a significant risk of bleeding, particularly intracranial, gastrointestinal, and retroperitoneal bleedings.

Although structurally diverse, all current anticoagulants either directly or indirectly target thrombin and/or factor Xa, two proteases that belong to the common pathway of coagulation. This is why these molecules are effective, yet it is also the reason that they cause internal bleeding. This disclosure focuses on new effective and safer anticoagulants that are associated with low risk of bleeding by inhibiting human factor XIIIa. All clotting enzymes, except one, are serine proteases. Factor XIIIa is a transglutaminase that catalyzes the last step in the coagulation process i.e., it is the only enzyme that acts downstream thrombin, a key enzyme for physiological and pathological blood clotting. This unique aspect has been under investigation in the context of venous thromboembolism. Factor XIIIa mechanically stabilizes the blood clot by cross-linking fibrin. Factor XIIIa also protects the clot from plasmin-mediated fibrinolysis by cross-linking α2-antiplasmin to fibrin. In fact, factor XIIIa is a major determinant of clot RBC content and clot size. Using venous thromboembolism model in inferior vena cava, thrombi from factor XIII deficient mice were found to have lower RBC content, reduced weight, and smaller size than those from wild type mice. Interestingly, mice carrying Ala mutations in fibrinogen residues γ390-396 exhibited decreased factor XIII binding to fibrinogen and delayed crosslinking. It also phenocopied FXIII-deficient mice, producing smaller venous thrombi with reduced RBC content. A specific factor XIIIa polymorphism was also found to substantially protect against venous thromboembolism & coronary artery disease. Such findings suggest that factor XIII(a)-fibrinogen axis is a novel therapeutic target for reducing venous thromboembolism. Notably, neither factor XIII-heterozygous nor Fibγ390-396A mice show signs of excessive bleeding.

A recent factor XIIIa experimental inhibitor delayed clot formation, reduced firmness, and facilitated clot lysis without affecting the clotting times indicating a little impact on hemostasis, in whole human blood using rotational thrombo-elastography. In a rabbit model of venous thromboembolism, the inhibitor decreased the weight of clots and facilitated flow restoration without prolongation of the bleeding time, which further validated factor XIIIa as a novel target for effective and safer anticoagulation. Stieler et al. (2020) ChemMedChem 15(10)L 900-905; Pasternack et al., (2020) J Thromb Haemost 18(1): 191-200. Overall, a FXIIIa inhibitor will lead to a smaller and “soft” clot that is more susceptible to plasmin hydrolysis, with little impact on hemostasis. Thus, FXIIIa inhibition is a new paradigm for developing new effective anticoagulants with a reduced or no risk of bleeding. Interestingly, a recent study showed that factor XIIIa-mediated cross-linking may contribute to the formation of amyloid β deposits in cerebral amyloid angiopathy and Alzheimer's disease. Hur et al. (2019) J Biol Chem 294(2)L: 390-396. Therefore, factor XIIIa can be pursued as a treatment or adjunct therapy for cerebral amyloid angiopathy and Alzheimer's disease.

FXIIIa inhibitors could also potentially be used clinically to reduce the incidence of acute kidney injury or acute lung injury in critically ill patients. Patients that were subjected to continuous renal replacement therapy could also benefit, either in regular hemodialysis or in the intensive care unit. The few small molecule factor XIIIa inhibitors available inhibit factor XIIIa with poor potency and/or potency against other transglutaminases. The phenyl unsaturated ketones with the general structure in Formula 1 described herein are potent and selective human factor XIIIa inhibitors for treatment of thromboembolic diseases and others. Thromboembolic diseases include but are not limited to diseases and conditions that involve thrombosis.

The Factor XIIIa inhibitors described herein can be used in a method for treating a condition selected from the group consisting of thrombosis, thromboembolism, complications from chronic kidney disease, complications in acute coronary syndrome, Alzheimer's disease, cerebral amyloid angiopathy, acute kidney injury, acute lung injury, coronary artery disease, or a combination thereof comprising administering an effective amount of the Factor XIIIa inhibitors described herein.

The Factor XIIIa inhibitors described herein can be used in a method for treating a condition selected from the group consisting of thrombosis, thromboembolism, complications from chronic kidney disease, complications in acute coronary syndrome, Alzheimer's disease, cerebral amyloid angiopathy, acute kidney injury, acute lung injury, coronary artery disease, or a combination thereof comprising administering a composition comprising effective amount of the Factor XIIIa inhibitors described herein.

### Factor XIIIa Inhibitors

The Factor XIIIa inhibitors described herein can be synthesized in high yields using well established chemistry. The Factor XIIIa inhibitors described herein were tested using fluorescence-based, bisubstrate-based trans-glutamination assay to determine IC₅₀ values. Their physiological relevance was established by a host of other assays. The Factor XIIIa inhibitors described herein can be used as orally bioavailable small molecules as anticoagulants for treatment of blood clotting diseases as well as Alzheimer's disease and other pathologies in which factor XIIIa is involved.

The Factor XIIIa inhibitors described herein can comprise the compound of Formula I:

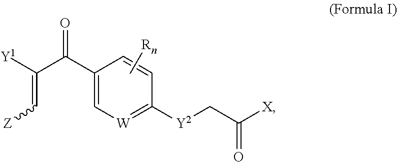

wherein R is —H, -halogen, C₁-C₆ alkyl, C₃-C₆ cycloalkyl, —OR³ (wherein R³ is C₁-C₆ alkyl), -acyl, or -benzoyl;
n is an integer of 1 to 3;
Y¹ is C₁-C₁₂ alkyl, or H;
Y² is —CH₂—, CH—(CH₃), C(CH₃)₂, NH, N(R⁴) (wherein R⁴═C₁-C₆ alkyl), O, S, or C═O;
X is —OH, OR⁵ (wherein R⁵═C₁-C₆ alkyl), NH₂, NHR₆ (wherein R⁶═C₁-C₆ alkyl), NR⁷R⁸ (wherein R⁷ and R⁸, independently, are C₁-C₆ alkyl), -halogen, or C₁-C₆ alkyl;
Z is H, —Cl, or C₁-C₆ alkyl (including E and Z-isomers); and
W is C, N, N-alkyl (wherein R⁹═C₁-C₆ alkyl), O, or S.

The Factor XIIIa inhibitors described herein can comprise compound of Formula II:

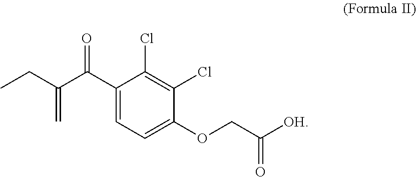 [SMILES: C=C(CC)C(=O)c1ccc(OCC(=O)O)c(Cl)c1Cl]

The Factor XIIa inhibitors described herein can comprise compound of Formula III:

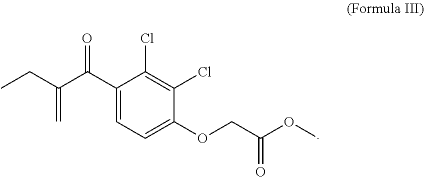

The Factor XIIIa inhibitors described herein can comprise compound of Formula IV:

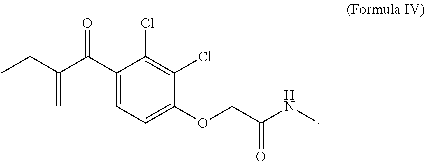

The Factor XIIIa inhibitors described herein can comprise compound of Formula V:

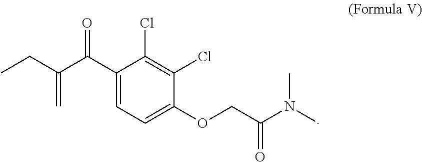

The Factor XIIIa inhibitors described herein can comprise compound of Formula VI:

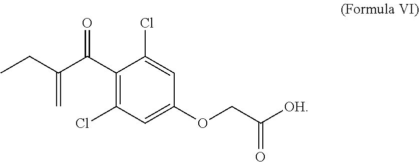 [SMILES: C=C(CC)C(=O)c1c(Cl)cc(OCC(=O)O)cc1Cl]

The Factor XIIIa inhibitors described herein can comprise compound of Formula VII:

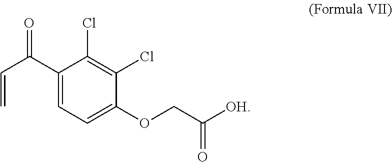 [SMILES: C=CC(=O)c1ccc(OCC(=O)O)c(Cl)c1Cl]

The Factor XIIIa inhibitors described herein can comprise compound of Formula VIII:

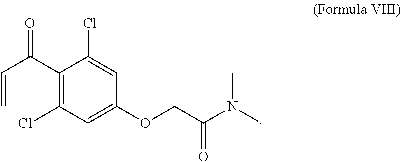

TABLE 1
Formulae and chemical names and formula

| Inhibitor | | |
|---|---|---|
| (Compound of) | Chemical name | Formula |
| Formula II | 2-(2,3-dichloro-4- | C₁₃H₁₂Cl₂O₄ |
| | (2-methylenebutanoyl) | |
| | phenoxy)acetic acid | |
| Formula III | methyl 2-(2,3-dichloro-4- | C₁₄H₁₄Cl₂O₄ |
| | (2-methylenebutanoyl) | |
| | phenoxy)acetate | |
| Formula IV | 2-(2,3-dichloro-4- | C₁₄H₁₅Cl₂NO₃ |
| | (2-methylene butanoyl)phenoxy)- | |
| | N-methylacetamide | |
| Formula V | 2-(2,3-dichloro-4- | C₁₅H₁₇Cl₂NO₃ |
| | (2-methylene butanoyl)phenoxy)- | |
| | N,N-dimethylacetamide | |
| Formula VI | 2-(3,5-dichloro-4- | C₁₃H₁₂Cl₂O₄ |
| | (2-methylenebutanoyl) | |
| | phenoxy)acetic acid | |
| Formula VII | 2-(4-acryloyl- | C₁₁H₈Cl₂O₄ |
| | 2,3-dichlorophenoxy)acetic acid | |
| Formula VIII | 2-(4-acryloyl- | C₁₃H₁₃Cl₂NO₃ |
| | 3,5-dichlorophenoxy)-N,N- | |
| | dimethylacetamide | |

### Therapeutic Methods

The social and economic burden of VTE is enormous and demands better treatments. Although available anticoagulants are effective, yet they are associated with bleeding. Factor XIIIa plays a critical role in venous thromboembolism development, and its inhibition appears to have limited effect on hemostasis. Thus, factor XIIIa inhibitors described herein can fulfill the goal of anticoagulation without bleeding, which is expected to address multiple unmet medical needs for which current therapies are limited because of safety concerns.

For instance, the Factor XIIIa-based anticoagulants described herein may benefit patients with atrial fibrillation. Because of the fear of bleeding, >30% of these patients fail to receive anticoagulant prophylaxis and among those given anticoagulation therapy, up to 50% are ineffectively treated. Another special group of interest is patients with chronic kidney disease. They are high-risk patients for thrombosis and have platelet abnormalities which put them at higher risk of bleeding. Other patients who will likely benefit from factor XIIIa-based anticoagulants are (1) venous thromboembolism patients who are at elevated risk of recurrent thrombosis upon discontinuation of anticoagulant therapy and (2) acute coronary syndrome patients who need anticoagulant prophylaxis in addition to antiplatelet therapy.

The Factor XIIIa inhibitors described herein can be used in methods of treating thromboembolism, optionally venous thromboembolism or arterial thromboembolism, chronic kidney disease, acute coronary syndrome patients, or a combination thereof.

Factor XIIIa inhibitors could be used clinically to reduce the incidence of acute kidney injury in critically ill patients. Patients who are subjected to continuous renal replacement therapy can also benefit, either in regular hemodialysis or in the intensive care unit. They can be considered on top of standard of care because of the low bleeding risk with an additional efficacy benefit and ultimately better outcome.

Factor XIIIa can also benefit Alzheimer's patients because Factor XIIIa forms unique complexes with amyloid-beta (Aβ) and colocalizes with deposited Aβ in cerebral amyloid angiopathy which is the pathological hallmark of Alzheimer's disease. Coagulation factor XIIIa cross-links amyloid β into dimers and oligomers and to blood proteins. Factor XIIIa was immunohistochemically detected in postmortem human brain tissue.

### Dosages

The pharmaceutical compositions described herein are administered to a subject in a manner known in the art. The dosage administered will be dependent upon the age, health, and weight of the recipient, kind of concurrent treatment, if any, frequency of treatment, and the nature of the effect desired.

The Factor XIIIa inhibitors described herein may be present in any suitable amount within the pharmaceutical compositions described herein. Those of skill in the art can readily determine suitable concentrations of Factor XIIIa inhibitors described herein to include in the pharmaceutical compositions depending on various factors including dosage and route of administration. Pharmaceutical compositions useful in the present invention can contain a quantity of a Factor XIIIa inhibitors described herein in an amount effective to be active as a contraceptive.

The Factor XIIIa inhibitors described herein may be present in the pharmaceutical composition in an amount of at least 0.1 mg/mL, at least 0.5 mg/mL, at least 1 mg/mL, at least 1.5 mg/mL, at least 2 mg/mL, at least 5 mg/mL, at least 10 mg/mL or at least 15 mg/mL.

The Factor XIIIa inhibitors described herein may be present in the pharmaceutical composition in an amount of between about 1 and 1,000 mg. For example, the Factor XIIIa inhibitors described herein may be present in the pharmaceutical composition in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg.

The pharmaceutical compositions may be administered in a single daily dose, or the total daily dosage may be administered in divided doses of two, three, or four times daily. Doses maybe administered for one week, one month, or over the course of several months, 3, 6, 9 or 12 months, or intervals known in the art and determined to be clinically relevant. Doses may be continued throughout the life of the patient, or discontinues when clinical judgment warrants. The daily dosage of the formulations may be varied over a wide range from about 0.0001 to about 1,000 mg per patient, per day. The range may more particularly be from about 0.001 mg/kg to 10 mg/kg of body weight per day, about 0.1-100 mg, about 1.0-50 mg or about 1.0-20 mg per day for adults (at about 60 kg). Additionally, the dosages may be about 0.5-10 mg/kg per day, about 1.0-5.0 mg/kg per day, 5.0-10 mg/kg per day, or equivalent doses as determine by a practitioner, to achieve a serum concentration that is clinically relevant.

As a non-limiting example, treatment of humans or animals can be provided as a one-time or periodic dosage of the Factor XIIIa inhibitors described herein 0.0001 to about 1,000 mg per patient, per day. The range may more particularly be from about 0.001 mg/kg to 10 mg/kg of body weight per day, about 0.1-100 mg, about 1.0-50 mg or about 1.0-20 mg per day for adults (at about 60 kg). Additionally, the dosages may be about 0.5-10 mg/kg per day, about 1.0-5.0 mg/kg per day, 5.0-10 mg/kg per day or equivalent doses as determine by a practitioner, to achieve a serum concentration that is clinically relevant.

Specifically, the pharmaceutical compositions described herein may be administered at least once a week over the course of several weeks. The pharmaceutical compositions may be administered at least once a day over several weeks to several months to several years. The pharmaceutical compositions may be administered daily over a period of several days, several weeks, several months, or several years, until no longer needed or desired.

### Routes of Administration

Routes of administration and dosages of effective amounts of the pharmaceutical compositions comprising the Factor XIIIa inhibitors are described herein. The Factor XIIIa inhibitors described herein can be administered in combination with other pharmaceutical agents in a variety of protocols for effective therapy.

The present disclosure further relates to the administration of at least one of the Factor XIIIa inhibitors described herein by the following routes, including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarenal, intraretinal, intraspinal, intrasynovial, intrathoracic, intrauterine, intravesical, bolus, vaginal, rectal, buccal, sublingual, intranasal, iontophoretic means, or transdermal means. A composition formulated for oral administration may comprise the Factor XIIIa inhibitors described herein

### Pharmaceutical Compositions

Pharmaceutical compositions suitable for parenteral administration include aqueous and non-aqueous sterile injection solutions which may contain anti-oxidants, buffers, bacteriostats and solutes that render the formulation isotonic with the blood of the intended recipient; and aqueous and non-aqueous sterile suspensions which may include suspending agents and thickening agents. The formulations may be presented in unit-dose or multi-dose containers, sealed ampules and vials, and may be stored in a freeze-dried (lyophilized) condition requiring only the addition of the sterile liquid carrier, water for injections, immediately prior to use. Extemporaneous injection solutions and suspensions may be prepared from sterile powders, granules and tablets of the kind previously described.

For parenteral administration, sterile suspensions and solutions are desired. Isotonic preparations which generally contain suitable preservatives are employed when intravenous, administration is desired. The pharmaceutical compositions may be administered parenterally via injection of a pharmaceutical composition comprising Factor XIIIa inhibitors described herein dissolved in an inert liquid carrier. The term “parenteral,” as used herein, includes, but is not limited to, subcutaneous injections, intravenous, intramuscular, intraperitoneal injections, or infusion techniques.

Acceptable liquid carriers include, vegetable oils including but not limited to peanut oil, cotton seed oil, sesame oil or combinations thereof, as well as organic solvents including but not limited to solketal., glycerol formal. The pharmaceutical compositions may be prepared by dissolving or suspending Factor XIIIa inhibitors described herein in the liquid carrier such that the final formulation contains from about 0.005% to 30% by weight of a cyclic peptide described herein.

For oral administration in the form of a tablet or capsule, the Factor XIIIa inhibitors described herein may be combined with an oral, non-toxic pharmaceutically acceptable inert carrier including but not limited to ethanol, glycerol, water or combinations thereof. Moreover, when desired or necessary, suitable binders, lubricants, disintegrating agents, and coloring agents may also be incorporated into the mixture. Suitable binders include, without limitation, starch; gelatin; natural sugars including but not limited to glucose or beta-lactose; corn sweeteners; natural and synthetic gums including but not limited to acacia, tragacanth, or sodium alginate, carboxymethylcellulose; polyethylene glycol; waxes or combinations thereof. Lubricants used in these dosage forms include, without limitation, sodium oleate, sodium stearate, magnesium stearate, sodium benzoate, sodium acetate, sodium chloride, or combinations thereof. Disintegrators include, without limitation, starch, methyl cellulose, agar, bentonite, xanthan gum, or combinations thereof.

For oral administration, the pharmaceutical formulation also optionally contains a sweetener. Sweeteners include but are not limited to sucrose, fructose, sodium saccharin, sucralose (SPLENDA®), sorbitol, mannitol, aspartame, sodium cyclamate, and combinations thereof.

Aqueous suspensions, emulsions and/or elixirs for oral administration can be combined with various sweetening agents, flavoring agents, including but not limited to, but not limited to orange or lemon flavors, coloring agents, including but not limited to dye stuffs, natural coloring agents or pigments, in addition to the diluents including but not limited to water, glycerin and various combinations.

The pharmaceutical compositions described herein suitable for oral administration may be presented as discrete units including but not limited to capsules, dragées, cachets or tablets each containing a predetermined amount of the Factor XIIIa inhibitors described herein; as a powder or granules; as a solution or a suspension in an aqueous liquid or a non-aqueous liquid; or as an oil-in-water liquid emulsion or a water-in-oil emulsion, and as a bolus.

A tablet may be made by compression or molding, optionally with one or more accessory ingredients. Compressed tablets may be prepared by compressing, in a suitable machine, the Factor XIIIa inhibitors described herein in a free-flowing form including but not limited to a powder or granules, optionally mixed with a binder, lubricant, inert diluent, preservative, surface active or dispersing agent. Molded tablets may be made by molding, in a suitable machine, a mixture of the powdered Factor XIIIa inhibitors described herein moistened with an inert liquid diluent. The tablets may be optionally coated or scored and may be formulated so as to provide a slow or controlled release of the Factor XIIIa inhibitors described herein therein.

In addition, the pharmaceutical compositions comprising Factor XIIIa inhibitors described herein may be incorporated into biodegradable polymers allowing for sustained release of the Factor XIIIa inhibitors described herein. The biodegradable polymers and their uses are described in detail in Brem et al., 74 J. NEUROSURG. 441-46 (1991). Suitable examples of sustained-release compositions include semipermeable matrices of solid hydrophobic polymers containing a cyclic peptide described herein, which matrices are in the form of shaped articles, e.g., films, or microcapsules. Examples of sustained-release matrices include polyesters, hydrogels (including poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers including but not limited to the LUPRON DEPOT® (Tap Pharmaceuticals, Inc., Chicago, Ill.) (injectable microspheres composed of lactic acid glycolic acid copolymer and leuprolide acetate), and poly-D-(−)-3-hydroxybutyric acid.

It can be sometimes desirable to deliver the Factor XIIIa inhibitors described herein to the subject over prolonged periods of time, for periods of one week to one year from a single administration. Certain medical devices may be employed to provide a continuous intermittent or on demand dosing of a patient. The devices may be a pump of diffusion apparatus, or other device containing a reservoir of drug and optionally diagnostic or monitoring components to regulate the delivery of the drug. Various slow-release, depot or implant dosage forms can be utilized. A dosage form can contain a pharmaceutically acceptable non-toxic salt of the Factor XIIIa inhibitors described herein that has a low degree of solubility in body fluids, (a) an acid addition salt with a polybasic acid including but not limited to phosphoric acid, sulfuric acid, citric acid, tartaric acid, tannic acid, pamoic acid, alginic acid, polyglutamic acid, naphthalene mono- or di-sulfonic acids, polygalacturonic acid, or combinations thereof; (b) a salt with a polyvalent metal cation including but not limited to zinc, calcium, bismuth, barium, magnesium, aluminum, copper, cobalt, nickel, cadmium, combinations thereof, or with an organic cation formed from e.g., N,N′-dibenzyl-ethylenediamine or ethylenediamine; or (c) combinations of (a) and (b) e.g., a zinc tannate salt. Additionally, the Factor XIIIa inhibitors described herein or a relatively insoluble salt including but not limited to those just described, can be formulated in a gel, an aluminum monostearate gel with, e.g., sesame oil, suitable for injection. Salts include, but are not limited to, zinc salts, zinc tannate salts, pamoate salts, or combinations thereof. Another type of slow-release depot formulation for injection would contain a salt of the Factor XIIIa inhibitors described herein dispersed or encapsulated in a slow degrading, non-toxic, non-antigenic polymer including but not limited to a polylactic acid/polyglycolic acid polymer, including the formulations as described in U.S. Pat. No. 3,773,919. The Factor XIIIa inhibitors described herein or relatively insoluble salts thereof including but not limited to those described above can also be formulated in cholesterol matrix silastic pellets, particularly for use in animals. Additional slow-release, depot or implant formulations, e.g., gas or liquid liposomes are known in the literature. See, e.g., U.S. Pat. No. 5,770,222.

Other examples include provision of the Factor XIIIa inhibitors described herein to be administered by sustained release delivery system containing a biodegradable composition. The biodegradable composition may be composed of a biodegradable, water-coagulable, non-polymeric material and a biocompatible, non-toxic organic solvent that is miscible to dispersible in an aqueous medium. The delivery system may be implanted at an implant site causing the solvent to dissipate, disperse or leach from the composition into surrounding tissue fluid through a resulting microporous matrix.

The term “implant site” is meant to include a site, in or on which the non-polymeric composition is applied. Implantation or implant site can also include the incorporation of the pharmaceutical composition comprising at least one of the Factor XIIIa inhibitors described herein with a solid device. The pharmaceutical composition can be incorporated into a coating on a stent that is implanted into a subject. Additionally, other solid or biodegradable materials can be used as a substrate on which the pharmaceutical composition is applied. The coated material, comprising the pharmaceutical composition is then implanted, inserted or is adjacent to the subject or patient. The term “biodegradable” means that the non-polymeric material and/or matrix of the implant will degrade over time by the action of enzymes, by simple or enzymatically catalyzed hydrolytic action and/or by other similar mechanisms in the human body. By “bioerodible,” it is meant that the implant matrix will erode or degrade over time due, at least in part, to contact with substances found in the surrounding tissue fluids, cellular action. By “bioabsorbable,” it is meant that the non-polymeric matrix will be broken down and absorbed within the human body, by a cell, a tissue.

Non-polymeric materials that can be used in the composition generally are those that are biocompatible, substantially insoluble in water and body fluids, and biodegradable and/or bioerodible. The non-polymeric material is capable of being at least partially solubilized in a water-soluble organic solvent. The non-polymeric materials are also capable of coagulating or solidifying to form a solid implant matrix. The non-polymeric material is combined with a compatible and suitable organic solvent to form a composition that has the desired consistency ranging from watery to viscous to a spreadable putty or paste.

Suitable organic solvents are those that are biocompatible, pharmaceutically-acceptable, and will at least partially dissolve the non-polymeric material. The organic solvent has a solubility in water ranging from miscible to dispersible. Optionally, a pore-forming agent can be included in the composition to generate additional pores in the implant matrix. The pore-forming agent can be any organic or inorganic, pharmaceutically-acceptable substance that is substantially soluble in water or body fluid, and will dissipate from the coagulating non-polymeric material and/or the solid matrix of the implant into surrounding body fluid at the implant site.

The Factor XIIIa inhibitors described herein are capable of providing a local or systemic biological, physiological or therapeutic effect in the body of an animal. In formulating some pharmaceutical compositions described herein, the Factor XIIIa inhibitors described herein may be soluble or dispersible in the non-polymeric composition to form a homogeneous mixture, and upon implantation, becomes incorporated into the implant matrix. As the solid matrix degrades over time, the Factor XIIIa inhibitors described herein are capable of being released from the matrix into the adjacent tissue fluid, and to the pertinent body tissue or organ, either adjacent to or distant from the implant site, preferably at a controlled rate. The release of the Factor XIIIa inhibitors described herein from the matrix may be varied by the solubility of the Factor XIIIa inhibitors described herein in an aqueous medium, the distribution of the Factor XIIIa inhibitors described herein within the matrix, the size, shape, porosity, and solubility and biodegradability of the solid matrix. See e.g., U.S. Pat. No. 5,888,533. The amounts and concentrations of ingredients in the composition administered to the patient will generally be effective to accomplish the task intended.

The Factor XIIIa inhibitors described herein may be administered by bioactive agent delivery systems containing microparticles suspended in a polymer matrix. The microparticles may be microcapsules, microspheres or nanospheres currently known in the art. The microparticles should be capable of being entrained intact within a polymer that is or becomes a gel once inside a biological environment. The microparticles can be biodegradable or nonbiodegradable. Many microencapsulation techniques used to incorporate a bioactive agent into a microparticle carrier are taught in the art. See e.g., U.S. Pat. Nos. 4,652,441; 5,100,669; 4,438,253; and 5,665,428.

A preferred polymeric matrix will be biodegradable and exhibit water solubility at low temperature and will undergo reversible thermal gelation at physiological mammalian body temperatures. The polymeric matrix is capable of releasing the substance entrained within its matrix over time and in a controlled manner. The polymers are gradually degraded by enzymatic or non-enzymatic hydrolysis in aqueous or physiological environments. See e.g., U.S. Pat. No. 6,287,588.

Methods of preparing various pharmaceutical compositions with a certain amount of active ingredients are known, or will be apparent in light of this disclosure, to those skilled in the art. Methods of preparing said pharmaceutical compositions can incorporate other suitable pharmaceutical excipients and their formulations are known in the art.

Methods of preparing the pharmaceutical preparations described herein are manufactured in a manner that is known, including conventional mixing, dissolving, or lyophilizing processes. Thus, liquid pharmaceutical preparations can be obtained by combining the Factor XIIIa inhibitors described herein with solid excipients, optionally grinding the resulting mixture and processing the mixture of granules, after adding suitable auxiliaries, if desired or necessary.

### Example 1

### Synthesis of Formula I-VIII

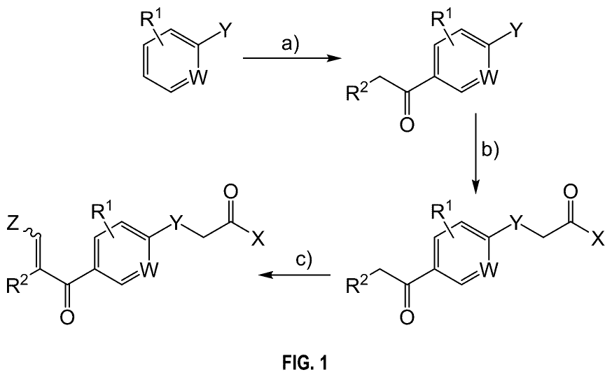

The synthesis of molecule with: disubstituted ring R═Cl—, Cl—; n is 2; Y¹═—CH₂CH₃; X═OH; and Y²═O was accomplished by a three-step scheme 1 (FIG. 1). The Friedel-Crafts acylation reaction of the dichloro-phenol with butanoyl chloride was performed in the presence of powdered aluminium chloride (AlCl₃) in carbon disulfide. Resulting molecule was purified by flash column chromatography on silica gel and consecutively refluxed in acetone for 48 hours in the presence of 1.2 equivalents of ethyl bromoacetate and two equivalents potassium carbonate (K₂CO₃). In the third step, an aldol condensation reaction, resulting intermediate was refluxed in an ethanol/water (50/50) mixture for 24 hours in the presence of two equivalents of formaldehyde and 2.5 equivalents of K₂CO₃. Final molecule was obtained by flash chromatography using a hexanes/ethyl acetate/methanol mixture as the eluent. FIG. 1.

NMR Data of compound of formula (II) in ppm (1H, 400 MHz, D2O): 1.065 (m, 3H), 2.37 (m, 2H), 4.62 (s, 2H), 5.70 (t, 1H), 6.08 (t, 1H), 6.91 (t, 1H), 7.26 (t, 1H). (13C, 400 MHz, D2O): 14.54, 25.81, 70.37, 113.55, 130.65, 134.64, 177.98, 202.31.

### Example 2

### Direct Inhibition of Human Factor XIIIA by Phenyl Unsaturated Ketones

To evaluate the effect of inhibitor on human factor XIIIa (and other transglutaminases), a bi-substrate, fluorescence-based trans-glutamination assay was performed. Generally, 1 μL of inhibitor was diluted with 87 μL of pH 7.4 buffer (50 mM Tris-HCl, 1 mM CaCl₂, 100 mM NaCl, and 2 mg/mL N,N-dimethylcasein) and 5 μL dithiothreitol (20 mM) at 37° C. followed by the addition of 2 μL of human factor XIIIa (0.3 μM) and incubation for 10 min. The activity of factor XIIIa was monitored following the addition of 5 μL of dansylcadaverine (2 mM) by measuring the initial rate of increase in fluorescence emission (λ_{Ex.}=360 nm and λ_{Em.}=490 nm). Relative residual factor XIIIa activity at each concentration of the inhibitor was calculated from the ratio of factor XIIIa activity in the presence and absence of the inhibitor. Logistic Eq. 1 was used to fit the concentration dependence of residual FXIa activity so as to obtain the potency (IC₅₀) and efficacy (ΔY %) of inhibition.

$$Y = {Y}_{0} + \frac{{Y}_{M} - {Y}_{0}}{1 + 1 {0}^{( {log [ I ]}_{0} - {logIC}_{50} ) ( H S )}} \qquad ( 1 )$$

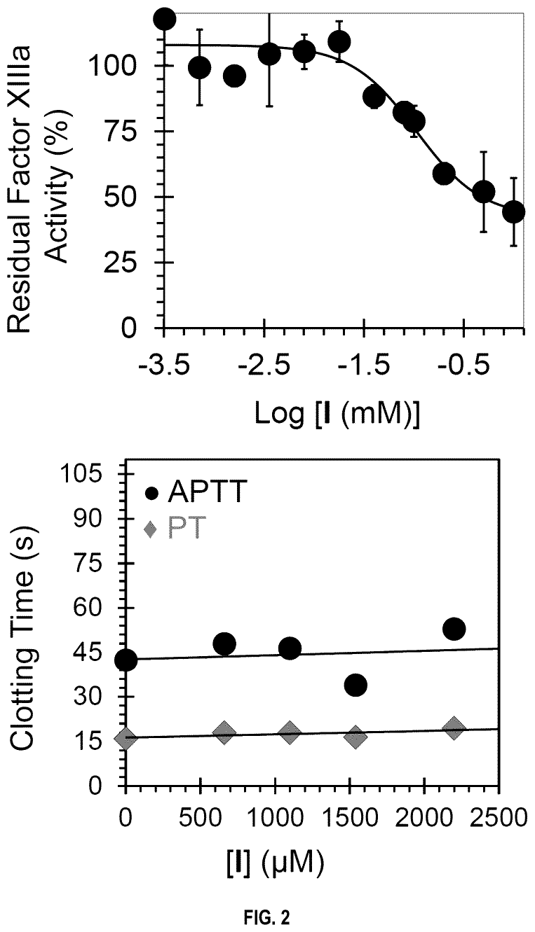

In this equation, Y is the ratio of residual FXIa activity in the presence of inhibitor to that in its absence, Y_{M} and Y₀ are the maximum and minimum possible values of the fractional residual factor XIIIa activity, IC₅₀ is the concentration of the inhibitor that leads to 50% inhibition of enzyme activity, and HS is the Hill slope. Y_{M}, Y₀, IC₅₀, and HS values are determined by nonlinear curve fitting of the data. The data is shown in FIG. 2 (top panel) and Table 1 below:

TABLE 1
Inhibition potency of Factor XIIIa by
phenyl unsaturated ketones (II-VIII)

| Inhibitor (Compound of) | FXIIIa IC₅₀ (μM) |
|---|---|
| Formula II | 105 |
| Formula III | 92 |
| Formula IV | 100 |
| Formula V | 75 |
| Formula VI | 51 |
| Formula VII | 83 |
| Formula VIII | 24 |

### Example 3

### Effect of Inhibitor on Human Plasma Clotting Times

The plasma clotting times; activated partial thromboplastin time (APTT) and prothrombin time (PT) were measured using the BBL Fibrosystem fibrometer (Becton-Dickinson, Sparles, MD). For the APTT assay, 10 μL of factor XIIIa inhibitor (0-2500 μM in the clotting cup) was mixed with 90 μL of citrated human plasma and 100 μL of prewarmed APTT reagent (0.2% ellagic acid). After incubation for 4 min at 37° C., clotting was initiated by adding 100 μL of prewarmed 25 mM CaCl₂, and the time to clotting was recorded. For the PT assay, thromboplastin-D was prepared according to the manufacturer's directions by adding 4 mL of distilled water, and then, the resulting mixture was warmed to 37° C. A 10 μL solution of factor XIIIa inhibitor (0-2500 μM in the clotting cup) was then mixed with 90 μL of citrated human plasma and was subsequently incubated for 30 sec at 37° C. Following the addition of 200 μL of prewarmed thromboplastin-D preparation, the time to clotting was recorded. In the two assays, about 5 or more concentrations of factor XIIIa inhibitor (0-2500 μM) were used to establish a concentration vs effect curve. The data were fit to a quadratic trendline, which was used to determine the concentration of factor XIIIa inhibitor necessary to double the clotting time as well as the other positive controls (dabigatran [thrombin inhibitor], rivaroxaban [FXa inhibitor], and AntiF11 [FXIa inhibitor]). Clotting times in the absence of factor XIIIa inhibitor were also determined in a similar fashion using 10 μL of highly purified water. The data is shown in FIG. 2 (bottom panel).

### Example 4

### Effect of Inhibitor on Factor XIIIA-Mediated Fibrin(Ogen) Polymerization

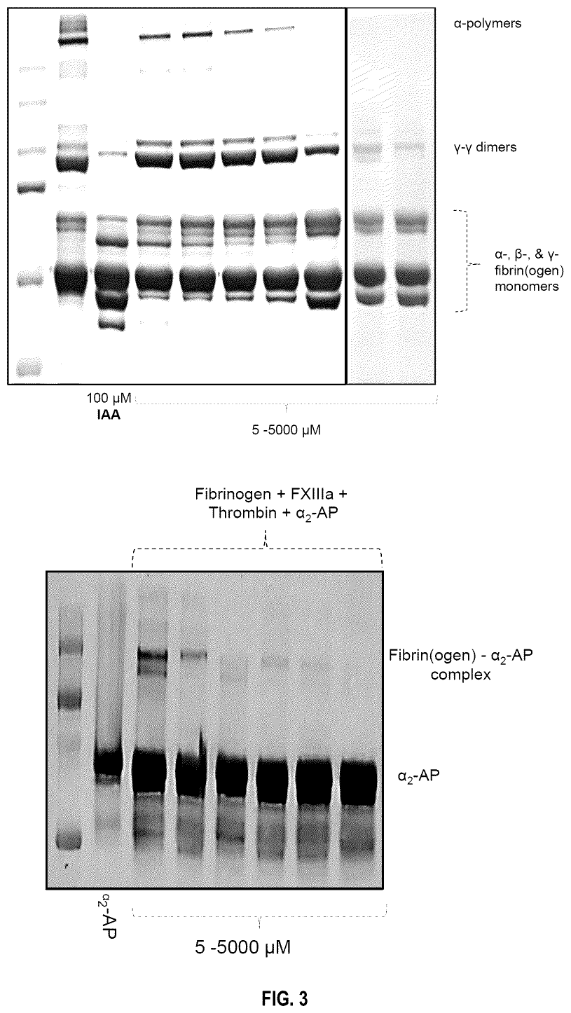

The effect of inhibitor on factor XIIIa-mediated fibrin polymerization was further investigated by gel electrophoresis. A solution containing 1.75 mg/ml fibrinogen and 0.9 μg/mL factor XIIIa in 50 mM Tris HCl buffer of pH 7.4 containing 10 mM CaCl₂ was incubated with different concentrations of inhibitor (5-5000 μM), and then clotted in the presence of human α-thrombin (1.25 μg/mL). The clots were incubated for 24 hrs at room temperature before the addition of denaturing buffer of 25 mM NaH₂PO₄, 5.7 M urea, 1.9% (w/v) SDS and 1.9% (w/v) DTT, and then incubated overnight at room temperature. Samples were boiled in a water bath for 10 min before centrifugation at 12000 g at 20° C. for 3 min; the supernatants were examined by SDS-PAGE on homogeneous 10% cross-linked gels and stained with Coomassie Brilliant Blue. The data is shown in FIG. 3 (top panel).

### Example 5

### Effect of Inhibitor on Factor XIIIA-Mediated Formation of Fibrin(Ogen)-A₂-AP Complex

This effect was investigated by western blot assay. A solution containing 1.75 mg/ml fibrinogen and 50 nM factor XIIIa in 50 mM HEPES buffer containing 5 mM CaCl₂ was incubated with different concentrations of inhibitor (100, 500, 1000, 3000, and 5000 μM). After incubation for 10 min, 1.25 μg/mL of thrombin was added to the concoction and further incubated for 30 min at room temperature. At the end of the incubation period, 4 μM of α₂-AP was added and then the reaction was quenched by using sample reducing buffer containing DDT. The mixture was then fractionated on 10% SDS-PAGE, and then transferred to nitrocellulose membrane, followed by blocking using 5% non-fat dry milk.

After vigorous wash with the washing buffer, the membrane was incubated with the primary antibody of human serpinF2/α₂-AP antibody from R&D systems. The secondary antibody was horseradish peroxidase conjugated anti-goat IgG from R&D systems. The relative positions of bands were confirmed using Western blot analysis. The data is shown in FIG. 3 (bottom panel).

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A compound of Formula I: 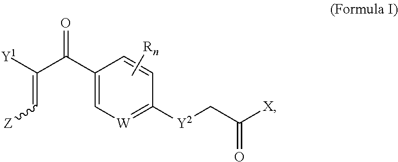
wherein, independently, R is —H, -halogen, C₁-C₆ alkyl, C₃-C₆ cycloalkyl, OR³ (wherein R³ is C₁-C₆ alkyl), -acyl, or -benzoyl;
n is an integer of 1 to 3;
Y¹ is C₁-C₁₂ alkyl, or —H;
Y² is CH₂—, CH—(CH₃), C(CH₃)₂, NH, N(R⁴) (wherein R⁴═C₁-C₆ alkyl), O, S, or C═O;
X is —OH, OR⁵ (wherein R⁵ is C₁-C₆ alkyl), NH₂, NHR⁶ (wherein R⁶ is C₁-C₆ alkyl), NR⁷R⁸ (wherein R⁷ and R⁸, independently, are C₁-C₆ alkyl), -halogen, or C₁-C₆ alkyl;
Z is H, —Cl, or C₁-C₆ alkyl (including E and Z-isomers); and
W is C, N, N(R⁹) (wherein R⁹ is C₁-C₆ alkyl), O, or S.

2. The compound of claim 1, wherein the compound is of Formula II:

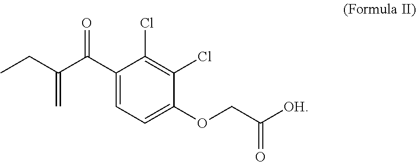 [SMILES: C=C(CC)C(=O)c1ccc(OCC(=O)O)c(Cl)c1Cl]

3. The compound of claim 1, wherein the compound is of Formula III:

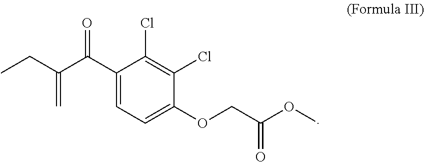

4. The compound of claim 1, wherein the compound is of Formula IV:

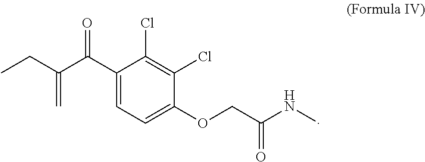

5. The compound of claim 1, wherein the compound is of Formula V:

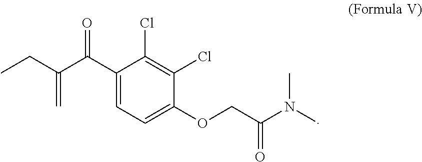

6. The compound of claim 1, wherein the compound is of Formula VI:

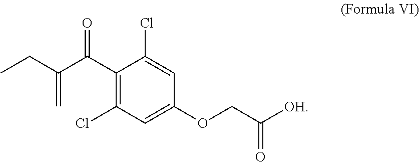 [SMILES: C=C(CC)C(=O)c1c(Cl)cc(OCC(=O)O)cc1Cl]

7. The compound of claim 1, wherein the compound is of Formula VII:

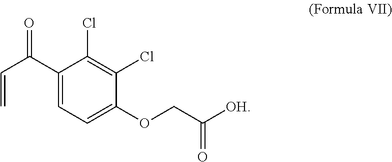 [SMILES: C=CC(=O)c1ccc(OCC(=O)O)c(Cl)c1Cl]

8. The compound of claim 1, wherein the compound is of Formula VIII:

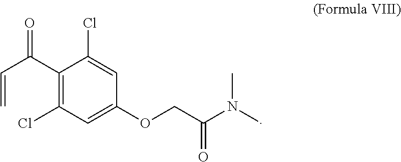

9. A composition comprising the compound of claim 1.

10. The composition of claim 9, wherein the composition is a pharmaceutical composition.

11. The composition of claim 9, wherein the composition further comprises a pharmaceutically acceptable excipient, carrier, diluent, adjuvant, vehicle, or a combination thereof.

12. A method for treating a condition selected from the group consisting of thrombosis, thromboembolism, complications from chronic kidney disease, complications in acute coronary syndrome, Alzheimer's disease, cerebral amyloid angiopathy, acute kidney injury, acute lung injury, coronary artery disease, or a combination thereof comprising administering an effective amount of the compound of claim 1 to a patient in need thereof.

13. The method of claim 12, wherein the compound or composition is administered intravenously, inhalation, subcutaneously, via infusion, orally, intrathecally, intraperitoneally, parenterally, or a combination thereof.

14. The method of claim 12, wherein the patient is a critically ill patient.

15. The method of claim 12, wherein the effective amount is between about 1 and 1,000 mg.

16. The method of claim 15, wherein the effective amount is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg.
